(19) United States
(12) Patent Application Publication
(10) Pub. No.: US 2025/0009554 A1
(43) Pub. Date: Jan. 9, 2025

(54) THERAPEUTIC EYE MASK WITH SLIDING EYE PODS

(71) Applicant: THE HILSINGER COMPANY PARENT, LLC, Mansfield, MA (US)

(72) Inventors: Alexander ARGO, Rio Grande (PR); Mark H. BRUDER, Alpharetta, GA (US)

(21) Appl. No.: 18/762,901

(22) Filed: Jul. 3, 2024

Related U.S. Application Data
(60) Provisional application No. 63/512,109, filed on Jul. 6, 2023.

(51) Int. Cl. A61F 7/02

(52) U.S. Cl. CPC A61F 7/02

(57) ABSTRACT

An example wearable mask includes a first coverage portion including a first front side and a first back side opposite the first front side. The first front side is configured to contact a first portion of a body of a user when the wearable mask is worn, and the first back side is configured to be directed away from the first portion when the wearable mask is worn. The first coverage portion further includes a first opening, a second opening, and a first passageway extending from the first opening to the second opening. The wearable mask further includes a strap extending through the first passageway. The first coverage portion is movably attached to the strap.

### CROSS-REFERENCE TO RELATED APPLICATIONS

This application claims the benefit of and priority to U.S. Provisional Patent Application No. 63/512,109, filed Jul. 6, 2023, entitled “THERAPEUTIC EYE MASK WITH SLIDING EYE PODS,” the content of which is hereby incorporated herein by reference in its entirety.

### TECHNICAL FIELD

Various embodiments of the present disclosure relate generally to therapeutic eye masks with one or more sliding eye pods and, more particularly, to therapeutic eye masks with one or more sliding eye pods that can deliver thermal therapy to a treatment region for a user.

### BACKGROUND

Eye conditions are fairly common among the general population and can include conditions such as dry eye syndrome, blepharitis, and Meibomian Gland Dysfunction (MGD), conjunctivitis, among others. Many of these conditions can benefit from improved blood flow, which can help reduce inflammation. However, many conventional eye treatment masks are not designed to adjustably and securely fit the eye areas of different users, causing issues in delivering therapeutic treatment for alleviating the above-mentioned conditions.

### SUMMARY OF THE DISCLOSURE

In some aspects, the techniques described herein relate to a wearable mask, comprising: a first front side and a first back side opposite the first front side, the first front side configured to contact a first portion of a body of a user when the wearable mask is worn, and the first back side configured to be directed away from the first portion when the wearable mask is worn; a first opening and a second opening; and a first passageway extending from the first opening to the second opening; and a strap extending through the first passageway, wherein the first coverage portion is movably attached to the strap.

In some aspects, the techniques described herein relate to a wearable mask, wherein at least the first coverage portion is slidably adjustable along a length of the strap for relocation to a desired location of the body for the user.

In some aspects, the techniques described herein relate to a wearable mask, wherein the first coverage portion is substantially oval, elliptical, polygonal, angled, or non-round in shape.

In some aspects, the techniques described herein relate to a wearable mask, wherein the strap comprises an adjustment mechanism that allows the user to adjust a length of the strap.

In some aspects, the techniques described herein relate to a wearable mask, wherein the adjustment mechanism comprises one or more of: snaps, ties, hook-and-loop fasteners, or other releasable attachment mechanisms.

In some aspects, the techniques described herein relate to a wearable mask, wherein the wearable mask is a therapeutic eye mask for thermal compressive treatment and/or delivery of medication to an eye of the user.

In some aspects, the techniques described herein relate to a wearable mask, wherein: the first coverage portion comprises an inner pouch containing a fill material, the inner pouch formed of a moisture-permeable material and configured to deliver heat and/or moisture; and the fill material is configured to absorb and release heat and/or moisture through the moisture-permeable material upon being heated.

In some aspects, the techniques described herein relate to a wearable mask, further comprising: a second coverage portion comprising a second front side and a second back side opposite the second front side, the second front side configured to contact a second portion of the body of the user when the wearable mask is worn and the second back side configured to be directed away from the second portion when the wearable mask is worn, the second coverage portion further comprising: a first opening and a second opening; and a second passageway extending from the first opening to the second opening, wherein the strap extends through the second passageway.

In some aspects, the techniques described herein relate to a wearable mask, wherein the second coverage portion is unmovably attached to the strap.

In some aspects, the techniques described herein relate to a wearable mask, wherein the second coverage portion is movably attached to the strap.

In some aspects, the techniques described herein relate to a wearable mask, wherein one or both of the first coverage portion and the second coverage portion are movably or slidably adjustable in position and/or spacing along a length of the strap to maintain a first length or spacing between the first coverage portion and the second coverage portion, and/or to allow adjustment of the spacing between the first and second coverage portions to fit a particular user.

In some aspects, the techniques described herein relate to a wearable mask, wherein: the first length is between about 1 to 36 inches; and the first length is less than the length of the strap.

In some aspects, the techniques described herein relate to a wearable mask, wherein the first coverage portion is removably attached to the strap.

In some aspects, the techniques described herein relate to a therapeutic mask, comprising: a plurality of coverage portions, each coverage portion of the plurality of coverage portions comprising: a front side and a back side opposite the front side, the front side configured to contact a first portion of a body of a user when the therapeutic mask is worn and the back side configured to be directed away from the first portion when the therapeutic mask is worn; an outer cover and an inner pouch, the inner pouch containing fill material configured to deliver heat when heated; a first opening and a second opening; and a passageway extending from the first opening to the second opening; and a strap extending through the first passageway, wherein at least one coverage portion of the plurality of coverage portions is movably attached to the strap.

In some aspects, the techniques described herein relate to a therapeutic mask, wherein each coverage portion is substantially oval, elliptical, polygonal, angled, or non-round in shape.

In some aspects, the techniques described herein relate to a therapeutic mask, wherein the strap comprises an adjustment mechanism that allows the user to adjust a length of the strap.

In some aspects, the techniques described herein relate to a therapeutic mask, wherein the adjustment mechanism comprises one or more of: snaps, ties, hook-and-loop fasteners, or other releasable attachment mechanisms.

In some aspects, the techniques described herein relate to a therapeutic mask, wherein: the plurality of coverage portions comprises a first coverage portion and a second coverage portion; and the first coverage portion and the second coverage portion are movably adjustable along a length of the strap to maintain a first length between the first coverage portion and the second coverage portion based at least in part on a location of one or more treatment areas for the user.

In some aspects, the techniques described herein relate to a therapeutic mask, wherein: the plurality of coverage portions comprises a first coverage portion and a second coverage portion; and the first coverage portion is movably attached to the strap and the second coverage portion is unmovably attached to the strap.

In some aspects, the techniques described herein relate to a therapeutic mask, wherein the strap comprises a flexible elastic material.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 depicts an example wearable mask with a pair of eye coverage portions according to one or more embodiments of the present disclosure.

FIG. 2 depicts the eye coverage portions that have been detached from the wearable mask shown in FIG. 1 according to one or more embodiments of the present disclosure.

FIG. 3 depicts a front view of the wearable mask shown in FIG. 1 according to one or more embodiments of the present disclosure.

FIGS. 4 and 5 depict a front view of the first back side of the first eye coverage portion of the wearable mask shown in FIG. 1 according to one or more embodiments of the present disclosure.

FIG. 6 depicts a perspective view of the wearable mask shown in FIG. 1 according to one or more embodiments of the present disclosure.

### DETAILED DESCRIPTION

Various embodiments of the present disclosure relate generally to therapeutic eye masks with sliding eye pods and, more particularly, to therapeutic eye masks with sliding eye pods that can deliver thermal (i.e., hot and/or cold) therapy to a treatment region for a user. Various conditions of the eye may require or benefit from medical or palliative care. For example, blepharitis is a common and ongoing condition where the eyelids become inflamed (swollen), with oily particles and bacteria coating the eyelid margin near the base of the eyelashes. This condition can cause irritation, itchiness, redness, dry eye and stinging or burning of the eyes.

An estimated 40.9 million people in the United States aged 18 or older wear contact lenses. Contact lens wearers often report dry eye symptoms and show signs of meibomian gland dysfunction (MGD) including gland atrophy, thinned lipid layer, and increased tear film instability. Contact lens wearers can also suffer from other eye conditions such as chalazion, styes (hordeolum), conjunctivitis, and eye fatigue. However, these conditions are not limited to just contact lens wearers, and the general population or former patients who have undergone eye surgery can also be affected by the above-mentioned conditions.

Many currently known eye treatment masks are not designed to securely and adjustably fit the eye area of a user, leading to ineffective therapeutic treatment. For example, conventional eye masks and eye compresses may not fit securely to the eye and/or lack adjustability functions of eye coverage portions, leading to poor fit for some segments of the population.

In the context outlined above, one or more embodiments of the present disclosure relate to a wearable mask that includes a first coverage portion or eye pod. The first coverage portion can include a first front side and a first back side opposite the first front side. The front side can be configured to contact a first portion of a body of a user when the wearable mask is worn, and the first back side can be configured to be directed away from the first portion when the wearable mask is worn. The first coverage portion can further include a first opening, a second opening, and a first passageway extending from the first opening to the second opening. The wearable mask can further include a strap extending through the first passageway, and the first coverage portion can be movably or slidably attached to the strap to allow selective adjustment of the position of the coverage portion on the strap.

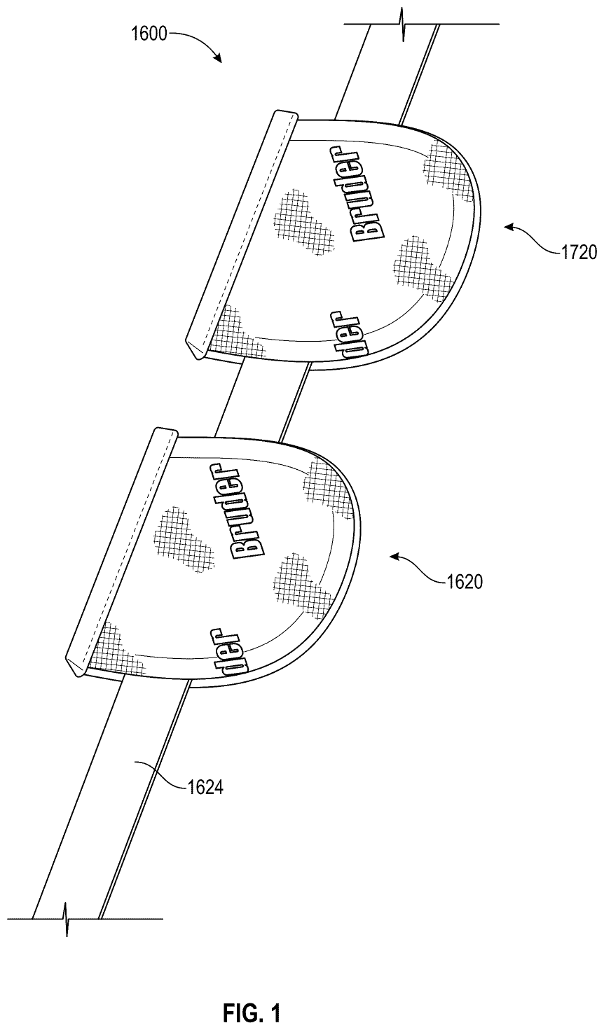

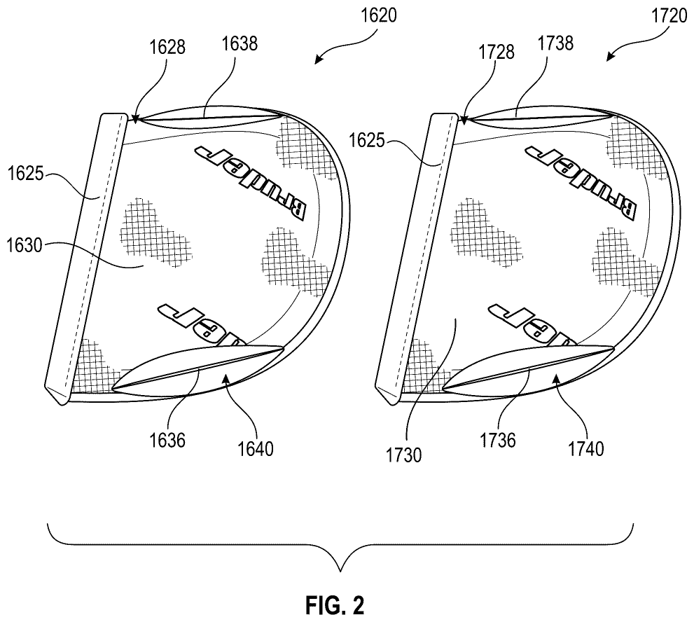

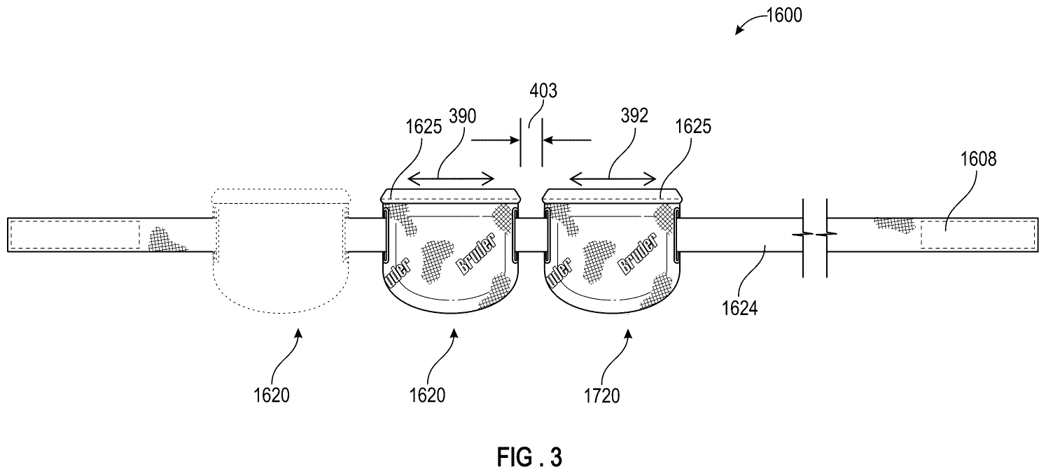

Turning to the drawings, FIG. 1 depicts an example wearable mask 1600 with two eye coverage portions, FIG. 2 depicts the eye coverage portions that have been detached from the wearable mask 1600, and FIG. 3 depicts a front view of the wearable mask 1600, according to one or more embodiments of the present disclosure. FIGS. 1-3 are not exhaustively illustrated, meaning that other components not shown in FIGS. 1-3 can be included or relied upon in some cases. Similarly, one or more components shown in FIGS. 1-3 can be omitted in some cases. Additionally, FIGS. 1-3 are not necessarily drawn to any particular scale or size. The wearable mask 1600 can be a therapeutic mask that can be used to treat various locations of a user's body. For example, the wearable mask 1600 can be used as a therapeutic eye mask for delivering compressive heat treatment or moist heat therapy to a user's (e.g., human or animal) eyes via sliding eye pods or sliding eye coverage portions. In other cases, the wearable mask 1600 can be used as a therapeutic mask for delivering compressive heat treatment or moist heat therapy to any other location of a body. One non-limiting example may include use of the wearable mask 1600 for mastitis where each eye coverage portion can be used separately for each breast of a user for delivering moist heat therapy.

The wearable mask 1600 can include a first eye coverage portion 1620 (e.g., a sliding eye pod), a second eye coverage portion 1720 (e.g., a sliding eye pod), and a strap 1624 extending through each of the first eye coverage portion 1620 and the second eye coverage portion 1720. The strap 1624 is a securing strap to secure the wearable mask 1600 to a desired location of a user. In this context, the strap 1624 includes an adjustment mechanism 1608 (FIG. 3) such as hook-and-loop attachment material, snaps, clips, and buttons, among others, to allow a user to adjust a length of the strap 1624 for securing the wearable mask 1600 to a desired location, for example to allow adjustment to fit comfortably around the head of a user and secure the eye coverage portions over the eyes of the user. The strap 1624 is also used to provide a sliding or moving mechanism to the first eye coverage portion 1620 and/or the second eye coverage portion 1720 about a length (e.g., horizontal length) of the strap 1624 as will be discussed in greater detail below. The strap 1624 may for example comprise a strip of fabric or other flexible material, and optionally may comprise an elastic material providing some degree of stretch to resiliently secure to the user's head or other body portion.

One or both of the first eye coverage portion 1620 and the second eye coverage portion 1720 include various openings and passageways to enable the strap 1624 to extend or pass through therebetween. For example, as shown in FIG. 2, the first eye coverage portion 1620 includes a first opening 1636 extending along a first edge of the first eye coverage portion 1620 and a second opening 1638 extending along a second edge of the first eye coverage portion 1620. The second edge is located opposite the first edge and the openings 1636 and 1638 are positioned to create a first passageway, slot or channel 1640 to enable the strap 1624 to fully pass through the first eye coverage portion 1620 via the first opening 1636 and the second opening 1638. Similarly, the second eye coverage portion 1720 includes a first opening 1736 and a second opening 1738 extending along a first edge of the second eye coverage portion 1720 and a second opening 1738 extending along a second edge of the second eye coverage portion 1720, with the second edge located opposite the first edge. The openings 1736 and 1738 are positioned to create a second passageway, slot or channel 1740 to enable the strap 1624 to fully pass through the second eye coverage portion 120 via the first opening 1736 and the second opening 1738.

Still referring to FIG. 2, the first eye coverage portion 1620 and the second eye coverage portion 1720 each includes a front side (not shown) configured to contact a portion of a user's body when the wearable mask 1600 is worn. Additionally, the first eye coverage portion 1620 and the second eye coverage portion 1720 each includes a back side opposite the front side and configured to be directed away from the portion of the user's body when the wearable mask 1600 is worn. The front side or inner side may include a smoother or softer material without coloration or printing, whereas the outer or back side may include a more durable or rougher textured material having branding text or logos, or other coloration or indicia imprinted or otherwise applied thereon. For example, instructional information may be imprinted or otherwise applied on one or both sides (for example, “this side toward you” printed on the inner or front side).

In particular, the first eye coverage portion 1620 includes a first back side 1628, and the second eye coverage portion 1720 includes a second back side 1728. Each back side 1628 and 1728 can include an outer cover that facilitates movement of the strap 1624 through the passageways 1640 and 1740. For example, the first back side 1628 includes a first outer cover 1630, and the second back side 1728 includes a second outer cover 1730. The outer covers 1630 and 1730 are generally identical and can be constructed of a lightweight, durable, and flexible material such as foam and polyester to aid in the delivery and retention of moist heat via the front side of each eye coverage portion 1620 and 1720. The material and construction of the front sides and the back sides 1628 and 1728 including the outer covers 1630 and 1730 are discussed in greater detail in the following paragraphs.

Referring now to FIG. 3, the first eye coverage portion 1620 and/or the second eye coverage portion 1720 are slidably attached to the strap 1624. For example, in one embodiment, the first eye coverage portion 1620 and the second eye coverage portion 1720 can be slidably attached to the strap 1624 to allow for selective repositioning of the eye coverage portion(s) along the strap 1624 to provide adjustment of the spacing between the first and second eye coverage portions 1620 and 1720 to fit users of different spacing between their eyes. To illustrate, the eye coverage portions 1620 and 1720 can be repositioned or slidably adjusted as indicated by directional arrows 390 and 392. In another embodiment, only one of the first eye coverage portion 1620 or the second eye coverage portion 1720 can be slidably adjustable relative to the strap 1624 as indicated by the directional arrows 390 and 392. Particularly, the first eye coverage portion 1620 can be slidably adjustable relative to the strap 1624 while the second eye coverage portion 1720 is permanently affixed or stitched to a predetermined location of the strap 1624, or vice-versa.

As illustrated in FIG. 3, a user can adjust a length or spacing 403 between the first eye coverage portion 1620 and the second eye coverage portion 1720. As discussed above, in one embodiment, this adjustment process may include physically sliding just the first eye coverage portion 1620 along a length (e.g., horizontal length) of the strap 1624 as indicated by the directional arrow 390, while the second eye coverage portion 1720 is unmovably attached to the strap 1624. For example, the first eye coverage portion 1620 may maintain a first position as indicated by the solid outline representation and after repositioning or sliding a distance from the first position along the length of the strap 1624, the first eye coverage portion 1620 may maintain a second position as indicated by the dotted outline representation depicted to the left of the solid outline representation. In another embodiment, a user may adjust the length 403 by physically moving or sliding both eye coverage portions 1620 and 1720 along a length of the strap 1624 as indicated by the directional arrows 390 and 392. In yet another embodiment, a user may adjust the length 403 by physically moving or sliding just the second eye coverage portion 1720 as indicated by the directional arrow 392, while the first eye coverage portion 1620 remains unmovably attached to the strap 1624. A user may wish to adjust the length 403 to better match the distance between one's eyes or other the distance between other treatment locations. In example embodiments, the length or spacing 403 may be adjustable between about 0 to 36 inches, and in further example embodiments between about 0.5 to 2 inches, and for less than a total length of the strap 1624. This adjustability mechanism can accommodate portions of the population who may have a wider or narrower distance between one's eyes compared to an average person. The sliding mechanism of the eye coverage portions 1620 and/or 1720 can accommodate a wide range of the population who need customizability for applying the wearable mask 1600 for therapeutic purposes.

The eye coverage portions 1620 and 1720 are independent structures such that each can independently conform to a respective eye region of a user. For example, each of the eye coverage portions 1620 and 1720 can have a radially curved lower or bottom profile, and a generally rectangular upper or top profile, defining a generally U-shaped configuration. In some embodiments, each of the eye coverage portions 1620 and 1720 can be substantially oval, elliptical, polygona, angled, or non-round in shape. Optionally, an upper attachment strip 1625 can be stitched along a top edge of each of the eye coverage portions 1620 and 1720. Alternatively, the eye coverage portions 1620 and 1720 can be combined in a single panel or mask format configured to extend over both eyes of a user. In the depicted embodiments, each of the eye coverage portions 1620 and 1720 can be formed from a series of layers or lamina.

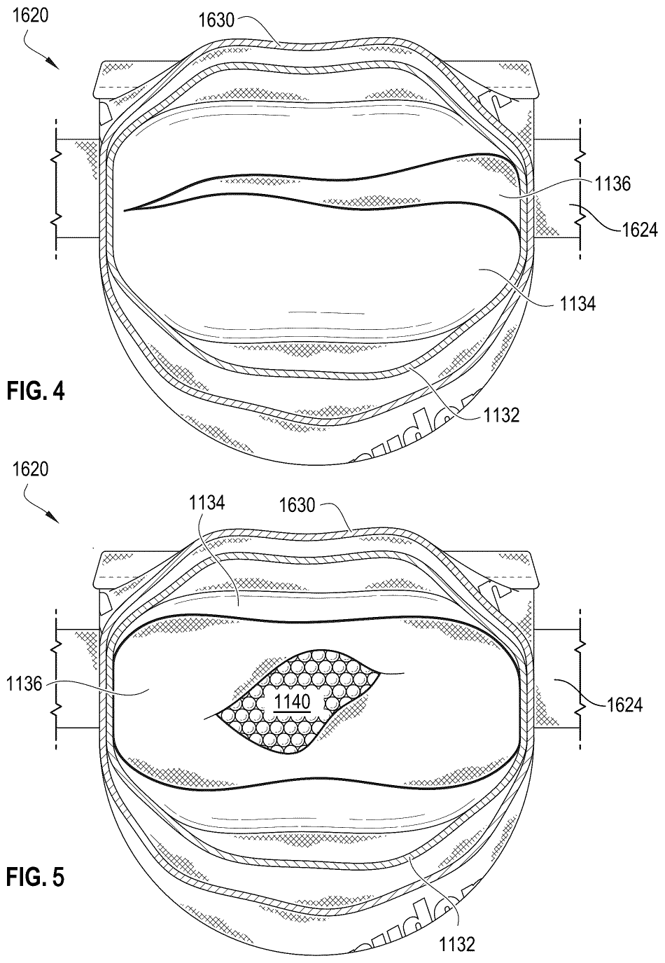

FIGS. 4 and 5 depict a front view of the first back side 1628 of the first eye coverage portion 1620 according to one or more embodiments of the present disclosure. Although FIGS. 4 and 5 depict the first back side 1628 of the first eye coverage portion 1620, the following description is applicable to the second back side 1728 of the second eye coverage portion 1720. FIGS. 4 and 5 are not exhaustively illustrated, meaning that other components not shown in FIGS. 4 and 5 can be included or relied upon in some cases. Similarly, one or more components shown in FIGS. 4 and 5 can be omitted in some cases. Additionally, FIGS. 4 and 5 are not necessarily drawn to any particular scale or size. The first eye coverage portion 1620 can further include an intermediate layer 1132 in between an inner pouch 1136 and the outer cover 1630. The intermediate layer 1132 is generally formed from a moisture permeable material. In example embodiments, the intermediate layer 1132 surrounds and contains the inner pouch 1136. In other embodiments, the intermediate layer 1132 covers only a portion of the inner pouch 1136. The inner pouch 1136 can be configured to contain a fill material 1140 (FIG. 5) which can retain moisture and deliver heat. For example, the fill material 1140 can include synthetic porous crystalline granular aluminosilicate zeolite—e.g., a hydrophilic natural or synthetic zeolite, also referred to as a molecular sieve material—or other substances with similar properties. The fill material 1140 can also include a multiplicity of individual solid beads or granules that absorb and release moisture while generally maintaining their individual solid shape and consistency. By way of example, a user can place the wearable mask 1600 in a microwave oven to heat or activate the fill material 1140 so that the fill material 1140 delivers moist heat through the front side of the first eye coverage portion 1620. In other example embodiments, the eye coverage portions may be self-heating, for example, by means of an exothermic reaction to activate the fill material and deliver moisture and/or thermal therapy to the treated body portion of the user.

The fill material 1140 may further include other inert additives and physical matrices without affecting the antimicrobial and hydrous efficacies of the fill material 1140. The hydrophilic zeolite granules or beads are configured to repeatedly absorb and release moisture without substantially changing shape or form. Optionally, the fill material 1140 can include a granular material such as activated alumina, silica gel, bentonite or hydrophilic zeolite or molecular sieve material loosely contained in the pouch or other enclosure. In alternate embodiments, the fill material 1140 can include capsules or packets of non-granular material (e.g., gel, liquid), powder, or other materials. The fill material 1140 optionally also contains a metallic or other antimicrobial agent, such as for example silver, copper, copper oxide, gold, magnesium oxide, aluminum oxide, titanium dioxide, zinc oxide, cobalt, nickel, zirconium, molybdenum, tin, lead and/or other metals; metal oxides, metal ions, metal particles or nanoparticles; and alloys, mixtures or combinations thereof deposited therein. For example, silver or other metal loading of the fill material 1140 may be attained by the process of ion-exchange. In this process, a solution containing atomic silver, or a composition of silver, bathes or is passed through a bed of the fill granules. An ion-exchange column method may be performed in which an aqueous solution containing atomic silver or a composition of silver may be passed through a column bed of the fill granules, and the eluted solution may again be passed through the bed or may receive additional silver and then be again passed through the bed.

Various ion-exchange schedules as known in the art may be applied to produce retention of the silver or other metals in the fill material 1140. For example, the final content by weight of an atomic silver or silver composition, or other metals or antimicrobial agents, may be as high as twenty percent of the final loaded fill granules. In example embodiments, the loaded fill granules produced by ion-exchange can exhibit high retention of the silver or other metals even under subsequent exposure to fluids and microwave irradiation. The fill granules may include a blend of both metal loaded and unloaded (i.e., not containing metal) zeolite or other substance(s) retaining silver or other metals. The presence of the atomic silver or other metals preferably will not interfere with the useful properties of the fill granules such as the moisture desorption, absorption, and/or adsorption properties which may be desirable in the use of the eye mask or compress system. The hydrophilic nature of example forms of zeolite fill materials provides that substantial water content is available therein and readily replenished by absorption of moisture in the form of water vapor from the atmosphere or ambient surroundings at standard room temperatures and conditions. The water so absorbed may be sufficient for moist heat delivery applications, or may be supplemented by manually added water, for providing a microwave responsive water content of the eye mask or compress system. The compositions of silver or other metals used may include but are not limited to, metal compounds, and metal salts such as silver chloride and silver nitrate.

The presence of silver or other metals within the fill granules, while optional, may provide anti-microbial properties to the wearable mask 1600. The ion-exchange loaded fill granules can preferably retain the silver or other metals despite microwave heating as may be required in the use of the eye mask or compress system. Further, the retention of the silver or other metals within the fill granules provides assured antimicrobial performance in a reusable and potentially washable, if so desired, moist heat therapy compress. In example embodiments, silver or other metals can be incorporated into the cover material (e.g., the outer cover 1630) and/or other portions of the wearable mask 1600, in addition to or instead of the fill granules. Alternatively, one or more non-metal antimicrobial materials and/or medications may optionally be incorporated into the fill material 1140, the eye coverage portions 1620 and 1720, and/or other portions of the wearable mask 1600.

Additionally, the first eye coverage portion 1620 can include a moisture barrier panel 1134 surrounding or covering at least a portion, for example the back side, of the inner pouch 1136. The moisture barrier panel 1134 is configured to prevent moisture from escaping from the back side 1628 of the first eye coverage portion 1620, thereby directing moist heat therapy toward the front or inner side of the first eye coverage portion 1620, for application to a treatment region of the user (i.e., in a user-treatment direction) by way of activation of the fill material 1140.

In example embodiments, the outer covers 1630 and 1730, the inner pouch 1136, and the intermediate layer 1132 can be constructed of a lightweight, durable, and flexible material such as foam or polyester. The material can be configured to allow moist heat to reach the face of a user but limit the amount of moisture released from the fill material 1140. In some embodiments, the outer covers 1630 and 1730, the inner pouch 1136, and/or the intermediate layer 1132 can be formed from an open cell thermoplastic polyurethane foam that is about 2 mm thick. In other examples, the outer covers 1630 and 1730, the inner pouch 1136, and/or the intermediate layer 1132 can be constructed from a 2 lb/ft³ (pounds per cubic foot) and ⅜″ thick polyether polyurethane foam. The foam layers can include polyester fabric laminated onto one or more sides.

Alternatively, the outer covers 1630 and 1730, the inner pouch 1136, and/or the intermediate layer 1132 can be constructed from polyester, rayon, spandex, silk, polyethylene, neoprene, ECA, ethylene-vinyl acetate (EVA), other plastic films, and/or other natural and/or synthetic fabrics or materials having the same or substantially similar characteristics and capabilities. For example, one or more of the layers of the outer covers 1630 and 1730, the inner pouch 1136, and/or the intermediate layer 1132 can be formed from polyester felt or from a closed cell foam such as polyurethane and/or thermoformable foam. The outer covers 1630 and 1730 can alternatively be constructed from a woven material different from the inner pouch 1136 and the intermediate layer 1132. The wearable mask 1600 can optionally include material having insulative or heat-transmissive properties to affect the temperature transferred from the mask 1600 to a user's eyes, better ensuring safety. Optionally, a recessed area may be formed at or around the center of the eye coverage portions, as by stitching or other attachment means between the front and back panels, to reduce heat transfer to the cornea regions of the eyes.

The wearable mask 1600 may be washable for reuse, or alternatively can be a single-use disposable product. In example embodiments, the eye coverage portions 1620 and 1720 can be constructed of a material containing nanobeads comprising an antimicrobial metal, medications, and/or other therapeutic material(s). Optionally, the mask 1600 may be configured for use in connection with medicated or therapeutic sheets, pods or other therapeutic accessories positioned or retained between the eye coverage portions 1620 and 1720 and the user's treatment areas. For example, the mask 1600 may be configured for supplemental delivery and transport of medication or therapeutic material with moist heat.

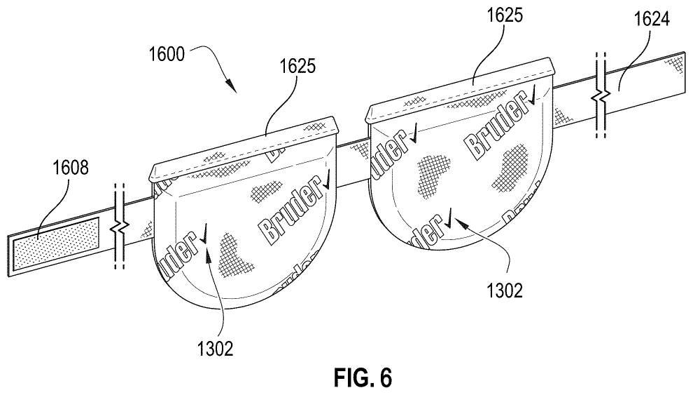

FIG. 6 depicts a perspective view of the wearable mask 1600 according to one or more embodiments of the present disclosure. FIG. 6 is not exhaustively illustrated, meaning that other components not shown in FIG. 6 can be included or relied upon in some cases. Similarly, one or more components shown in FIG. 6 can be omitted in some cases. Additionally, FIG. 6 is not necessarily drawn to any particular scale or size. The wearable mask 1600 can include thermochromic ink printed on each of the eye coverage portions 1620 and 1720. For example, the eye coverage portions 1620 and 1720 can include a checkmark 1302 that indicates whether the wearable mask 1600 is safe for application to a face of a user. Preferably, the thermochromic ink includes a customizable temperature range with ±1° C. of accuracy. The thermochromic ink can be formulated for screen printing, ultraviolet (UV), and flexographic applications. When the temperature of the thermochromic ink exceeds the recommended application temperature, the checkmark 1302 can disappear. In other embodiments, one or more thermally responsive indicators of various formats may be applied to a thermal therapy compress or other treatment device to indicate a temperature that is either within a specified range, or outside of a specified range (for example an indicator stating “TOO HOT/COLD-DO NOT USE” or the like when outside of a specified temperature range, and/or “SAFE TEMPERATURE FOR USE” or the like when within a specified temperature range), for example by printing in thermochromic ink or other thermally responsive mode of application. Alternatively, or in addition, thermally responsive color indicators (e.g., green for use, red for do not use, etc.) may be applied.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims. If a component is described as having “one or more” of the component, it is understood that the component can be referred to as “at least one” component.

The terms “first,” “second,” etc. are used only as labels, rather than a limitation for a number of the objects. It is understood that if multiple components are shown, the components may be referred to as a “first” component, a “second” component, and so forth, to the extent applicable.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

The above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A wearable mask, comprising:
a first coverage portion comprising:
a first front side and a first back side opposite the first front side, the first front side configured to contact a first portion of a body of a user when the wearable mask is worn, and the first back side configured to be directed away from the first portion when the wearable mask is worn;
a first opening and a second opening; and
a first passageway extending from the first opening to the second opening; and
a strap extending through the first passageway, wherein the first coverage portion is movably attached to the strap.

2. The wearable mask of claim 1, wherein the first coverage portion is slidably adjustable along a length of the strap for relocation to a desired location of the body for the user.

3. The wearable mask of claim 1, wherein the first coverage portion is substantially oval, elliptical, polygonal, angled, or non-round in shape.

4. The wearable mask of claim 1, wherein the strap comprises an adjustment mechanism that allows the user to adjust a length of the strap.

5. The wearable mask of claim 4, wherein the adjustment mechanism comprises one or more of: snaps, ties, hook-and-loop fasteners, or other releasable attachment mechanisms.

6. The wearable mask of claim 1, wherein the wearable mask is a therapeutic eye mask for thermal compressive treatment and/or delivery of medication to an eye of the user.

7. The wearable mask of claim 1, wherein:
the first coverage portion comprises an inner pouch containing fill material, the inner pouch formed of a moisture-permeable material and configured to deliver heat; and
the fill material is configured to absorb and release moisture through the moisture-permeable material upon being heated.

8. The wearable mask of claim 1, further comprising:
a second coverage portion comprising a second front side and a second back side opposite the second front side, the second front side configured to contact a second portion of the body of the user when the wearable mask is worn and the second back side configured to be directed away from the second portion when the wearable mask is worn, the second coverage portion further comprising:
a first opening and a second opening; and
a second passageway extending from the first opening to the second opening,
wherein the strap extends through the second passageway.

9. The wearable mask of claim 8, wherein the second coverage portion is unmovably attached to the strap.

10. The wearable mask of claim 8, wherein the second coverage portion is movably attached to the strap.

11. The wearable mask of claim 8, wherein the first coverage portion and the second coverage portion are movably adjustable along a length of the strap to maintain a first length between the first coverage portion and the second coverage portion.

12. The wearable mask of claim 11, wherein:
the first length is between about 1 to 36 inches; and
the first length is less than the length of the strap.

13. The wearable mask of claim 1, wherein the first coverage portion is removably attached to the strap.

14. A therapeutic mask, comprising:
a plurality of coverage portions, each coverage portion of the plurality of coverage portions comprising:
a front side and a back side opposite the front side, the front side configured to contact a first portion of a body of a user when the therapeutic mask is worn and the back side configured to be directed away from the first portion when the therapeutic mask is worn;
an outer cover and an inner pouch, the inner pouch containing fill material configured to deliver heat when heated;
a first opening and a second opening; and
a passageway extending from the first opening to the second opening; and
a strap extending through the passageway, wherein at least one coverage portion of the plurality of coverage portions is movably attached to the strap.

15. The therapeutic mask of claim 14, wherein each coverage portion is substantially oval, elliptical, polygonal, angled, or non-round in shape.

16. The therapeutic mask of claim 14, wherein the strap comprises an adjustment mechanism that allows the user to adjust a length of the strap.

17. The therapeutic mask of claim 14, wherein the adjustment mechanism comprises one or more of: snaps, ties, hook-and-loop fasteners, or other releasable attachment mechanisms.

18. The therapeutic mask of claim 14, wherein:
the plurality of coverage portions comprises a first coverage portion and a second coverage portion; and
the first coverage portion and the second coverage portion are movably adjustable along a length of the strap to maintain a first length between the first coverage portion and the second coverage portion based at least in part on a location of one or more treatment areas for the user.

19. The therapeutic mask of claim 14, wherein:
the plurality of coverage portions comprises a first coverage portion and a second coverage portion; and
the first coverage portion is movably attached to the strap and the second coverage portion is unmovably attached to the strap.

20. The therapeutic mask of claim 14, wherein the strap comprises a flexible clastic material.
